(19) United States
(12) Plant Patent Application Publication
(10) Pub. No.: US 2015/0121590 P1
(43) Pub. Date: Apr. 30, 2015

(54) Vriesea plant named 'V858-2'

(50) ;

(71) Applicant: Herb Hill, Lithia, FL (US)

(72) Inventor: Herb Hill, Lithia, FL (US)

(21) Appl. No.: 13/998,402

(22) Filed: Oct. 28, 2013

(51) Int. Cl. A01H 5/00

(52) U.S. Cl. PLT/263.1

(57) ABSTRACT

A new and distinct Vriesea cultivar named ‘V858-2’ is disclosed, characterized by well-branched flaming orange-red inflorescence color. The new variety is a Vriesea, typically produced as an indoor ornamental plant.

### LATIN NAME OF THE GENUS AND SPECIES

Vriesea hybrid

### VARIETY DENOMINATION

‘V858-2’

### BACKGROUND OF THE INVENTION

This application relates to a new cultivar of Vriesea. The new variety is the product of a planned breeding program. The new variety originated as a seedling from the crossing of the unpatented, proprietary seed parent Vriesea ‘v.478-1’ with the unpatented, proprietary pollen parent referred to as Vriesea ‘v.668-3’. The crossing was made by the inventor in his research greenhouse in Lithia, Fla. in October of 2003.

The new variety was first selected by the inventor, Herb Hill, a citizen of United States, in April of 2006 in a research greenhouse belonging to the inventor in Lithia, Fla. After identifying the new variety as a potentially interesting selection, the inventor continued confidential testing and propagation of ‘V858-2’, assessing stability of the unique characteristics of this variety.

Asexual reproduction of the new cultivar ‘V858-2’ was first performed at a commercial laboratory in Kudelstaart, Netherlands by tissue culture in 2008. Access to all plants was restricted, as plants were kept in a greenhouse not open to the public. Through subsequent propagation, multiple generations have been reproduced, which have shown that the unique features of this cultivar are stable and reproduced true to type.

### SUMMARY OF THE INVENTION

The cultivar ‘V858-2’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘V858-2.’ These characteristics in combination distinguish ‘V858-2’ as a new and distinct Vriesea cultivar:

1. Flaming orange-red inflorescence.

2. Green leaves.

3. Well branched inflorescence.

### PARENT COMPARISON

Plants of the new cultivar ‘V858-2’ are similar to the seed parent in most horticultural characteristics. However the new variety, ‘V858-2’ differs from the seed parent in the following characteristics:

1. Shorter inflorescence.

Plants of the new cultivar ‘V858-2’ are similar to the pollen parent in most horticultural characteristics. However the new variety, ‘V858-2’ differs from the pollen parent in the following characteristics:

1. Different colored inflorescence.

### COMMERCIAL COMPARISON

‘V858-2’ is similar in most horticultural characteristics to the unpatented commercial variety Vriesea ‘Energy’ U.S. Plant Pat. No. 18,235 in most horticultural characteristics. However the new variety, ‘V858-2’ differs from ‘Energy’ in the following characteristics:

1. Inflorescence of ‘V858-2’ branches heavier than ‘Energy’.

2. Color pattern of inflorescence is flamed in ‘V858-2’ whereas ‘Energy’ is plain.

### BRIEF DESCRIPTION OF THE PHOTOGRAPH

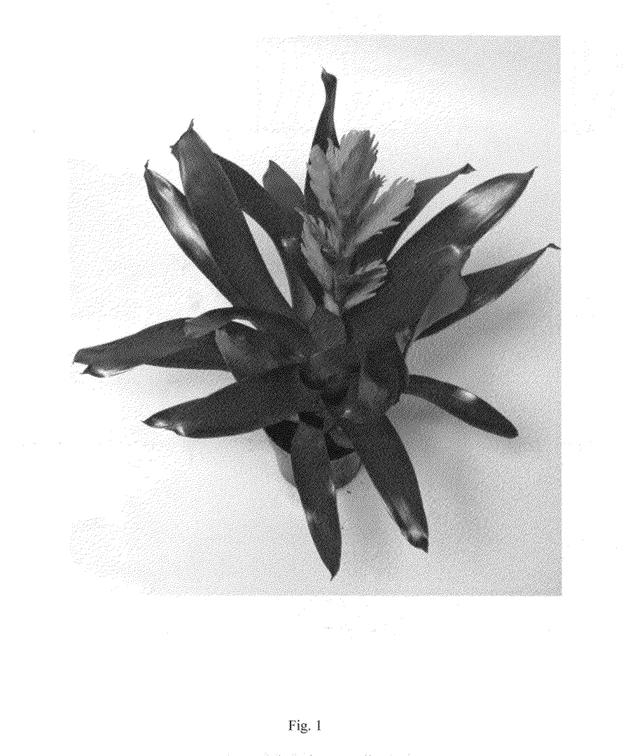

The accompanying photograph in FIG. 1 illustrates in full color a typical plant of ‘V858-2’ grown in a commercial greenhouse in De Kwakel, Netherlands. The plant is 20 months old from a rooted cutting, in a 12 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe a typical plant of ‘V858-2’ grown in a commercial greenhouse in De Kwakel, Netherlands. The plant is 20 months old from a rooted cutting, in a 12 cm pot. Temperatures ranged between 20° C. and 28° C. during the day 18° C. and 22° C. at night. No photoperiodic or chemical treatments were given to the plants. No supplemental lighting was given. Measurements and numerical values represent averages of typical plant types.
Botanical classification: Vriesea hybrid ‘V858-2’
Propagation:
   Type of propagation typically used.—Tissue culture.
   Time to initiate roots.—About 15 days at approximately 20° C.
   Time to produce a rooted cutting/liner.—About 180 days at approximately 22° C.
   Root description.—Densely branched, non-fibrous, non-fleshy, colored Greyed-orange; near RHS 165A.
Plant:
   Growth habit.—Main rosette with inflorescence growing from the centre; overall plant-shape: broad ovate.
   Height.—Approximately: 38.0 cm.
   Plant spread.—Approximately: 41.5 cm.
   Normal pot size.—12 cm circular pot.
   Growth rate.—Moderate.
   Branching characteristics.—No stems, no lateral branches present, plant consists of a basal rosette.
   Number of leaves per stem.—Approximate number of leaves per rosette (no stems present): 27.
Foliage:
   Leaf.—Arrangement: Rosulate (in a basal rosette) Average Length: Approximately 26.0 cm. Average Width: Approximately 4.0 cm (measured at 50% of the leaf blade). Shape of blade: Ligulate Aspect: Leaves in an average angle of 60° to horizontal (=0°) Apex: Abruptly acute, moderately to strongly twisted, tip slightly to moderately downward in an average angle of −80° to horizontal (=0°) Base: Sheathing Sheath length: Average 7.4 cm Sheath width: Average 7.4 cm. Sheath color: Upper side purple; in between near RHS N77C and N79B, underside purple; in between near RHS N77C and N79A Margin: Entire Texture of top surface: Smooth, very glossy Texture of bottom surface: Smooth, very glossy Color: Young foliage upper side: Green; near RHS N 137A Young foliage under side: Yellow-green; in between near RHS 146A and 147B Mature foliage upper side: Green to yellow-green; in between near RHS N137A and 147A but much darker Mature foliage under side: Green; near RHS N137B Venation: Type: Parallel Venation coloration: As leaf blade Petiole: No petiole present.
Inflorescence:
   General description.—Scape growing from the centre of the basal rosette, inflorescence branched into a paniculate shape; only bracts visible, individual flowers not visible but fully enclosed by bracts. Inflorescence Height, excluding scape: Approximately 25.9 cm. Inflorescence Width: Approximately 8.8 cm.
   Scape.—Length: Approximately 29.3 cm. Width: Approximately 0.8 cm. Aspect: Approximately 90° angle to rosette (=0°) Strength: Strong Color: Red; near RHS 51A.
   Bracts.—
   Scape/lowermost bracts.—Shape: Ovate Length: Approximately 2.7 cm, excluding sheath. Width: Approximately 1.8 cm (measured at the base, just above the sheath) Quantity: Average 13. Tip: Abruptly acute Base: Sheath length: Approximately 3.0 cm Sheath width: Approximately 2.8 cm. Sheath color: Red; near RHS 53D Margin: Entire Color Upper side: Lower bracts yellow-green; near RHS 146A and 146B. apex greyed-purple; near N186C, upper bracts red; near 46A, apex yellow-green; near RHS 146A Color Under side: Lower bracts green; near RHS 143A, apex greyed-purple; near 187C, upper bracts red; near RHS 47A, tinged yellow-green, near RHS 146C, apex yellow-green; near RHS 146A.
   Terminal bracts.—Shape: Ovate, folded (flattened) Length: Approximately 2.3 cm, excluding sheath. Width: Approximately 2.8 cm; however, bracts are folded, so in effect it is two times 1.4 cm. Quantity: Average 120 Tip. Apiculate Base: Sheathing Sheath length: Approximately 2.3 cm. Sheath width: Approximately 3.0 cm; however, bracts are folded, so in effect it is two times 1.5 cm. Sheath color: Orange-red; near RHS N30A Margin: Entire Coloration Terminal Bracts: Upper side: Orange; near RHS 24B, fading yellow-orange towards the margins; near RHS 14C Under side: Orange-red; near RHS 34A, fading yellow-orange towards the margins; near RHS 21B.
Flower:
   Flowering characteristics.—Individual flowers fully enclosed by the bracts and therefore not visible from outside. ; each bract encloses one individual flower.
   Days to flowering from tissue culture.—2 to 2.5 years.
   Inflorescence and flower type and habit.—Inflorescence paniculate, individual flower single.
   Flower longevity on plant.—Average: 3 months.
   Quantity of buds and flowers per plant.—Average: 120.
   Persistent or self-cleaning.—Persistent.
   Bud.—Shape: Lanceolate Length: Average: 1.7 cm Diameter: Average: 0.4 cm Color: Yellow-green; near RHS 150B to 150C.
   Flower size.—Diameter: Average: 0.6 cm. Depth: Average: 2.7 cm.
   Petals.—Quantity: Average: 3 Arrangement: Rotate, fused Length: Average: 1.7 cm Width: Average: 0.5 cm Shape: Oblong Apex: Obtuse Base: Broad cuneate, fused Margin: Entire Texture, upper and lower surfaces: Glabrous, moderately glossy Color: When opening, upper surface: Yellow; near RHS 4C, apex yellow-green; near RHS 150A When opening, lower surface: Yellow; near RHS 4A, 4B and 4C, tip yellow-green; near RHS N144A Fully opened, upper surface: Yellow; near RHS 4C, apex yellow-green; near RHS 150A Fully opened, lower surface: Yellow; near RHS 4A, 4B and 4C tip yellow-green; near RHS N144A.
   Sepals.—Quantity: Average: 3 Length: Average: 2.5 cm Width: Average: 0.9 cm Shape (overall): Lanceolate Apex: Acute Base: Cuneate, fused Margin: Entire Color: Yellow; near RHS 10B, apex near RHS 8A.
   Peduncles.—Length: Average: 6.2 cm Diameter: Average diameter: 0.5 cm Angle: About 25° to the lateral branch Strength: Strong Texture: Glabrous, glossy Color: Yellow-green; near RHS 150C.
   Fragrance.—No fragrance.
Reproductive organs:
   Stamen.—Number: Average: 6.
   Anthers.—Shape: Narrow oblong Length: Approximately 0.7 cm. Color: Yellow and yellow-orange; near RHS 13A and 12B Pollen: No pollen detected.
   Pistil.—Number: Average: 1 Length: Average: 0.7 cm. Style: Length: Average: 0.65 cm. Color: Yellow-green; near RHS 150D, top darker; near RHS 151B Stigma: Shape: Club-shaped Color: Yellow-green; near RHS 150B Ovary Color: Yellow-green; near RHS 150D.
Other characteristics:
   Disease resistance.—Neither resistance nor susceptibility to the normal diseases and pests of Vriesea has been observed.
   Temperature tolerance.—Observed to tolerate temperatures to 35° C. without any negative effects. Temperature tolerance above this range has not been observed. Tolerance of cold temperatures has not been observed.
   Fruit/seed production.—No fruits/seeds detected to date

What is claimed is:

1. A new and distinct cultivar of Vriesea plant named ‘V858-2’ as herein illustrated and described.
